(19) United States
(12) Patent Application Publication
(10) Pub. No.: US 2011/0293543 A1
(43) Pub. Date: Dec. 1, 2011

(54) ANHYDROUS COMPOSITION COMPRISING A DIBENZOYLMETHANE COMPOUND AND A COLOR PIGMENT

(76) Inventor: Wei Yu, Edison, NJ (US)

(21) Appl. No.: 12/788,859

(22) Filed: May 27, 2010

(51) Int. Cl. A61K 8/18; A61Q 17/04; A61Q 1/02

(52) U.S. Cl. 424/59

(57) ABSTRACT

The invention relates to an anhydrous color cosmetic composition including a cosmetically-acceptable hydrophobic vehicle that includes a wax component, an oil component, and an organic UV filter component that includes a dibenzoylmethane compound. The composition also includes a powder component that includes a color pigment and an oil-absorbing powder.

### FIELD

The present invention relates to a color cosmetic composition, such as a cosmetic composition including a dibenzoylmethane and a color pigment.

### BACKGROUND OF THE INVENTION

Color cosmetics are used to impart color to the skin, especially the face. Often, it is desirable to include additives, such as sunscreens, in color cosmetics, to provide additional benefits to the skin. However, in order to achieve high levels of protection it is necessary to include organic sunscreens. In particular, to achieve high levels of UV-A protection, it is often desirable to use a dibenzoylmethane compound, e.g., avobenzone.

Unfortunately, it is known that combining avobenzone and color pigments into a single composition can cause problems such as reactivity between the two, and degradation of avobenzone.

Applicant has now discovered that problems of instability associated with combinations of avobenzone and color pigments can be overcome. Specifically, the problem can be overcome by an anhydrous composition that includes a wax component, an oil component, a powder component including a color pigment, and an organic UV filter component that includes a dibenzoylmethane compound.

### SUMMARY OF THE INVENTION

The invention relates to an anhydrous color cosmetic composition comprising: (1) a cosmetically-acceptable hydrophobic vehicle comprising a wax component, an oil component, and an organic UV-filter component dissolved in the wax component and the oil component, wherein the organic UV-filter component comprises a dibenzoylmethane compound and is present in a concentration by weight of at least about 15% of the color cosmetic composition; and (2) a powder component distributed in and substantially insoluble in the hydrophobic vehicle, the powder component comprising a color pigment present in a concentration by weight of at least about 0.1% of the color cosmetic composition, and an oil-absorbing powder.

### DETAILED DESCRIPTION OF THE INVENTION

Unless defined otherwise, all technical and scientific terms used herein have the meaning commonly understood by one of ordinary skill in the art to which the invention pertains. All publications, patent applications, patents, and other references mentioned herein are incorporated by reference. Unless otherwise indicated, a percentage refers to percentage by weight (i.e., % (W/W)) of the inventive color cosmetic composition.

As used herein, “safe and effective amount” means an amount sufficient to provide a desired benefit at a desired level, but low enough to avoid serious side effects.

As used herein, the term “treating” or “treatment” means alleviation or elimination of symptoms, cure, prevention, or inhibition of a human condition or disease, specifically of the skin.

As used herein the term “substantially free of” means that the composition includes less than about 1% of the species in question, preferably less than about 0.5%, more preferably less than about 0.1%, and even more preferably is completely free of the particular species.

Compositions of the present invention are useful for application to the skin, especially the face, including lips, eyelids, and eyelining and other body surfaces on which color cosmetic products are typically used. In certain preferred embodiment, the composition is used as a so-called “foundation,” a color cosmetic product which is generally applied across the entire face (e.g., via brush, pad, sponge, or similar porous applicator) and upon which additional cosmetics may optionally be applied. In another preferred embodiment, the composition is used as a “concealer,” typically used prior to applying foundation, for example for concealing age or acne spots or scars. In yet another embodiment, the compositions is used as a lip treatment. In yet another embodiment, the composition is used as a “primer,” for application directly to the skin underneath foundations and/or concealers. Primers ease the application of foundation (or other color cosmetic composition) onto the skin, even out skin tone, and increase the longevity of color cosmetic compositions applied over the primer. Primers also may be used to smooth fine lines, such as around the mouth. A lip primer used underneath lipstick can maintain lip color and prevent feathering of the lipstick.

Compositions of the present invention are “anhydrous.” As used herein, “anhydrous” means the compositions include less than about 0.25% water. In a preferred embodiment, the compositions comprise less than about 0.1% of water. In yet a further preferred embodiment, the compositions are completely free of water. These maximum levels of water may, in certain embodiments refer only to “free” water, i.e., water that is not chemically bound to another compound (e.g., in a crystal as water of hydration).

### Cosmetically-Acceptable Hydrophobic Vehicle

Compositions of the present invention include a cosmetically-acceptable hydrophobic vehicle. As used herein, “cosmetically-acceptable” means suitable for use in contact with tissues (e.g., the skin) without undue toxicity, incompatibility, instability, irritation, allergic response, or the like. This term is not intended to limit the composition it describes as for use solely as a cosmetic (e.g., the composition may be used as a pharmaceutical).

The cosmetically-acceptable hydrophobic vehicle (“vehicle”) is generally suitable to serve as a medium or carrier by which the color pigment, the dibenzoylmethane, and other ingredients are uniformly spread across the skin of the user. The vehicle may provide other benefits such as emolliency, humectancy and surface tension reduction. As one skilled in the art will readily appreciate, the term vehicle is not meant to include other active ingredients or powder components, all of which are described subsequently in this specification.

The vehicle generally includes one or more solvents for the organic UV-filter component. In certain embodiments, a sufficient amount of one or more solvents for dibenzoylmethane is included in the vehicle to completely solublize the dibenzoylmethane at one or more temperatures in the range from 25° C. to 120° C.

The amount of vehicle in the composition may be varied depending upon, for example, the amount of spreadibility and lubriciousness, as well as the level of UV protection, desired in the composition. In certain embodiments, the amount of vehicle present in the composition ranges from about 20% to 75%, preferably from about 30% to about 70%, more preferably from about 40% to about 70%, most preferably from about 50% to about 70% by weight. In certain embodiments, vehicle consists essentially of, or consists of, an oil component and a wax component.

The cosmetically-acceptable hydrophobic vehicle includes a wax component. As used herein, “wax component” means one or more hydrophobic compounds that have a melting point (or melting range) from about 30° C. to about 120° C., preferably in the range of 45° C. to 100° C. In a preferred embodiment, the wax component includes a hydrophobic compound having a melting point from about 75° C. to 100° C.

As used herein, “hydrophobic compound” means a compound that includes a hydrophobic moiety meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is not amphiphilic and, and such, does not include hydrophilic moieties, such as anionic, cationic, zwitterionic, or nonionic group, that is polar, including sulfate, sulfonate, carboxylate, phosphate, phosphonates, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the hydrophobic compound does not include hydroxyl moieties.

Compounds suitable for use in or as the wax component include any of various hydrocarbons (straight or branched chain alkanes or alkenes, ketone, diketone, primary or secondary alcohols, aldehydes, sterol esters, alkanoic acids, turpenes, monoesters), such as those having a carbon chain length ranging from C₁₂-C₃₈. Also suitable are diesters or other branched esters. In a preferred embodiment, the compound is an ester of an alcohol (glycerol or other than glycerol) and a C₁₈ or greater fatty acid. Non-limiting examples include any of various natural waxes including beeswax (e.g., White Beeswax SP-422P available from Strahl and Pitsch, New York), insect waxes, sperm whale oil, lanolin, vegetable waxes such as canauba wax, jojoba oil, candelilla wax; mineral waxes such as paraffin wax; and synthetic waxes such as cetyl palmitate, lauryl palmitate, cetostearyl stearate, and polyethylene wax (e.g., PERFORMALENE 400, having a molecular weight of 400 and a melting point of 83-88° C., available from New Phase Technologies of Sugar Land, Tex.); and silicone waxes such as C30-45 alkyl methicone and C30-45 olefin (e.g., Dow Corning AMS-C30, having a melting point of 70° C., available from Dow Corning of Midland, Mich.). In certain embodiments, the wax component includes a high melting point ester of glycerol such as glycerol monostearate.

The amount of wax component present in the composition may be varied, but is generally suitable to reduce the fluidity of the composition drastically on cooling to room temperature, as well as, in conjunction with the oil component, solubilize the organic UV-filter component. In general, if the wax component only includes low melting point waxes, it is desirable to have higher concentrations of wax component in the composition. Similarly, if the wax component only includes high melting point waxes, it is desirable to have lower concentrations of wax component in the composition. In certain embodiments of the invention, the concentration of wax component in the composition is from 2% to about 20% by weight of the composition, preferably from about 4% to about 15%, most preferably from about 5% to about 12%. In certain other embodiments, the wax component has a weighted average melting point (averaged over all waxes in the wax component, each weighted by its relative concentration by weight) that is from about 55° C. to 85° C., such as from 60° C. to 80° C.

The cosmetically-acceptable hydrophobic vehicle also includes an oil component. As used herein, “oil component” means one or more hydrophobic compounds (such as those defined above with respect to wax component) that have a melting point that is below 30° C. Suitable examples of compounds useful in or as the oil component include vegetable oils (glyceryl esters of fatty acids, triglycerides) and fatty esters. Specific non-limiting examples include, without limitation, esters such as isopropyl palmitate, isopropyl myristate, isononyl isonanoate (such as WICKENOL 151 available from Alzo Inc. of Sayreville, N.J.), C₁₂-C₁₅ alkyl benzoates (such as FINSOLV TN), caprylic/capric triglycerides, silicone oils such as polydimethylsiloxane (dimethicone) and cyclopentasiloxane, and mineral oil.

As one skilled in the art will appreciate, for purposes of this disclosure, the term “oil component” does not include those compounds described below that are suitable for use in or as the organic UV-filter component.

The amount of oil component present in the composition may be varied, but is generally suitable to provide spreadability and pleasant skin-feel to the composition as well as, in conjunction with the wax component, solubilize the organic UV-filter component. In certain embodiments of the invention, the concentration of oil component in the composition is from 4% to about 40%, preferably from about 10% to about 40%, more preferably from about 10% to about 35%, and most preferably from about 20% to about 30% by weight of the composition.

The cosmetically-acceptable hydrophobic vehicle also includes an organic UV-filter component dissolved in said cosmetically-acceptable hydrophobic vehicle. The organic UV-filter component comprises one or more of UV filters suitable for cosmetic/topical use and commonly used to screen out ultraviolet radiation. The UV filters may include those compounds having appreciable absorbance in either or both the UV-A portion (320 nm to 400 nm) or in the UV-B portion (290 nm to 320 nm) of the ultraviolet spectrum. For example, when such a compound is cast into a film, it is possible to generate a molar extinction coefficient measured for at least one wavelength in the above wavelength ranges of at least about 1000 mol⁻¹ cm⁻¹, preferably at least about 2000 mol⁻¹ cm⁻¹, more preferably at least about 4000 mol⁻¹ cm¹. In a preferred embodiment, the molar extinction coefficient among at least 40% of the wavelengths in this portion of the spectrum is at least about 1000 mol⁻¹ cm⁻¹.

The organic UV-filter component comprises a dibenzoylmethane compound. As used herein, “dibenzoylmethane compound” means a UV-absorbing compound characterized by the following structure:

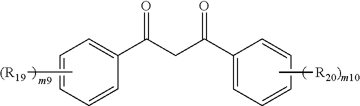

In a preferred embodiment, R₁₉ and R₂₀, independently, are C₁-C₈ alkyl or C₁-C₈ alkoxy groups, m9 is 0 to 3, and m10 is 1 to 3.

Examples and the synthesis of dibenzoylmethane compounds are disclosed in U.S. Pat. No. 4,489,057 and include, but are not limited to, 4-(1,1-dimethylethyl)-4′-methoxydibenzoylmethane (avobenzone and sold as PARSOL 1789, DSM), 2-2-methyldibenzoylmethane, 4-methyl-dibenzoylmethane, 4-isopropyldibenzoylmethane, 4-tert-butyldibenzoylmethane, 4-tert-butyl-4′-methoxydibenzoylmethane, 2,4-dimethylbenzoylmethane, 2,5-dimethylbenzoylmethane, 4,4′-diisopropylbenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and 2,6-dimethyl-4-tert-butyl-4′-methoxydibenzoylmethane.

Other non-limiting examples of UV filters that may be included in the UV-filter component are tertrahydroxybenzophenones; dicarboxydihydroxybenzophenones and alkane ester or acid halide derivatives thereof; dihydroxy-, dicarboxy-, and hydroxycarboxydibenzoylmethanes and alkane ester or acid halide derivatives thereof; dihydroxy-, dicarboxy-, and hydroxycarboxystilbenes and alkane ester or acid halide derivatives thereof; bis(hydroxystyrenyl) benzenes; bis(carboxystyrenyl)benzenes and alkane ester or acid halide derivatives thereof; dihydroxy-, dicarboxy, and hydroxycarboxycarotenes and alkane ester or acid halide derivatives thereof; 2 cyano-3,3-diphenyl acrylic acid, 2-ethyl hexyl ester. Other examples include UV-absorbing triazoles, i.e., those containing a five-membered heterocyclic ring with two carbon and three nitrogen atoms. Additional examples include 4-aminobenzoic acid and alkane esters thereof; anthranilic acid and alkane esters thereof; salicylic acid and alkane esters thereof; hydroxycinnamic acid alkane esters thereof; dihydroxy-, dicarboxy-, and hydroxycarboxybenzophenones and alkane ester or acid halide derivatives thereof; dihydroxy-, dicarboxy-, and hydroxycarboxychalcones and alkane ester or acid halide derivatives thereof; dihydroxy-, dicarboxy-, and hydroxycarboxycoumarins and alkane ester or acid halide derivatives thereof.

The organic UV-filter component may be a solid or liquid at room temperature. However, in order to provide a high degree of UV protection, the organic UV-filter component should be dissolved in the oil and wax components of the composition (e.g., the organic UV-filter component should not exist predominantly or entirely as a powder, crystalline or otherwise, in the composition). Dissolution of the organic UV-filter component in the wax and oil components is performed at elevated temperature.

The relative proportions of the UV filters in the organic UV-filter component may be varied, but in order to provide sufficient levels of UV-protection, the organic UV-filter component (total of all UV-filters) must be at least 15% by weight of the entire composition, preferably from about 20% to about 50% of the composition. As percentage by weight of the vehicle, the organic UV-filter component may range from about 20% to about 65% of the vehicle, preferably from about 35% to about 60%.

Furthermore, in order to provide sufficient UV-A protection, the dibenzoylmethane compound must be present in the composition in a concentration by weight of at least 1%, preferably at least 1.5% (e.g., from about 1.5% to about 4%), more preferably at least 2% (e.g., from about 2% to about 3.5%).

### Powder Component

Compositions of the present invention include a powder component distributed in and substantially insoluble in the hydrophobic vehicle. By “distributed in and substantially insoluble in the hydrophobic vehicle,” it is meant that the constituents of the powder component exist as particulates in the composition, typically having an average particle size of greater than about 0.1 microns.

The amount of powder component present in the composition may be varied, but is generally suitable to provide and distribute color and UV protection uniformly onto the skin, as well as absorb the oil component to reduce any unfavorable oily feel to the user. In certain embodiments of the invention, the concentration of powder component in the composition is from 15% to about 55%, preferably from about 22% to about 50%, more preferably from about 25% to about 45% by weight of the composition.

The powder component comprises a color pigment. As used herein, “color pigment” means a particulate material that does not dissolve in the vehicle (rather it remains in a particulate form in the composition) and imparts a non-white color to the skin. In one embodiment, the color pigment is a metal-containing color pigment. As used herein, “metal containing” means the pigment includes an elemental, ionic or a coordinated metal species. The amount of metal in the metal-containing color pigment may be at least about 5%, such as at least about 15% by weight of the metal-containing pigment.

The metal-containing color pigment may be a transition metal oxide, such as an iron oxide, (including red and yellow iron oxides), a chromium pigment and the like. In another embodiment the metal-containing color pigment is a sulfur-containing aluminum/sodium silicate (e.g., ultramarine, a deep blue pigment). In yet another embodiment the metal-containing color pigment is a lake pigment (i.e.; an organic dye such as azo, indigoid, triphenylmethane, anthraquinone, and xanthine dyes that are designated as D&C and FD&C blues, browns, greens, oranges, reds, yellows, etc., precipitated onto inert binders such as insoluble salts, such as an aluminum salt). In another embodiment, the metal-containing color pigment is an interference pigment such as one having a substrate (e.g., mica) that is coated with thin layer(s) of material having a higher refractive index than the substrate. Suitable coating materials include aluminum oxide, titanium dioxide, bismuth oxychloride, iron oxide, or salts of zirconium, cobalt, and the like.

In yet another embodiment, the color pigment is coated, such as with an organosilane or other surface modifier.

In order to impart sufficient color to the skin, the color pigment must comprise at least 0.1%, preferably at least 0.25%, most preferably at least about 0.5% by weight of the total composition. In certain embodiments, the color pigment is no more than about 5% by weight of the composition.

The powder component further includes an oil-absorbing powder. As used herein, “oil-absorbing powder” means a powder that has a mineral oil absorption capacity of at least about 20 ml/100 g. Mineral oil absorption capacity is determined by placing 3-5 grams of test sample on a glass plate and adding mineral oil one drop at a time and mixing with a spatula. The end point is indicated when the sample produces a very stiff paste which does not break or separate. The paste should be able be rolled with the spatula without separation. Testing is done in triplicate. The oil-absorbing powder may have a low to moderate oil absorption, such as from about 20 ml/100 g to about 60 ml/100 g.

The powder component may or may not also comprise filler pigments that provide opacity or other functionality. Suitable filler pigments include titanium dioxide, talc, mica, silica, boron nitride, and the like. The average particle size of the filler pigments may range from about 0.5 microns to about 20 microns. The filler pigments may be present in a concentration from about 15% to about 35%, such as from about 18% to about 30%, more preferably from about 20% to about 30% by weight of the composition. Specific examples of suitable filler pigments include titanium dioxide having a primary particle size of 0.2 micron (available as Atlas White AS R3435 from LCW Sensient Cosmetic Technologies of South Plainfield N.J.), silica having an average particle size of about 6 micron (available as Spheron EP-MMS from Presperse Incorporated of Somerset N.J.); mica having an average particle size of 4.7 micron (available as Sercite PHN from Presperse Incorporated of Somerset N.J.); and calcium aluminum borosilicate having an average particle size of 9-13 microns (available as Luxsil from Presperse Incorporated of Somerset N.J.).

The oil-absorbing powder may include a highly-oil-absorbing powder. A “highly-oil-absorbing powder” is one having a mineral oil absorption of greater than about 200 ml/100 g of sample. The average particle size of the highly-oil-absorbing powder may range from about 0.5 microns to about 20 microns, such as from about 2 microns to about 15 microns. Examples include silicas such as SILSPHERE LS-8H available from Argan Oil Products of Northridge, Calif.; and silicas such as MSS-500/H, MSS-500/3H and silica shell, all available from Kobo Products. The highly-oil-absorbing powders may be present in a concentration from about 1% to about 10%, such as from about 2% to about 10%, preferably from about 2% to about 6% by weight of the composition. The weight ratio of concentration of highly-oil-absorbing powder to powder component may be from about 0.05 to about 0.25, such as from about 0.1 to about 0.2.

The powder component may include non-oil-absorbing powders that may provide functionality such as slip or other manifestations of skin-feel. “Non-oil-absorbing powders” are those having a mineral oil absorption of less than about 20 ml/100 g of sample. Examples of non-oil-absorbing powders include calcium aluminum borosilciates and other glass particles such as LUXSIL, available from Presperse of Somerset, N.J. The non-oil absorbing powders may be present in a concentration from about 1% to about 10%, such as from about 1% to about 6% by weight of the composition.

The powder component may include organic polymeric particles (that, like other ingredients of the powder component, do not dissolve in the vehicle). Examples include durable acrylic particles such as copolymers of styrene and acrylates such as styrene/acrylates copolymer available as SUNSPHERES from Dow Chemical of Midland, Mich., having a diameter from about 1-20 microns.

The relative proportion of vehicle to powder component is preferably selected to provide a low degree of fluidity to the composition, yet still allow the composition to be easily applied to the skin. The weight ratio of vehicle to powder component may be from about 1:1 to about 3:1, such as from about 1.5:1 to about 2.5:1, such as from about 1.5:1 to about 2.2:1. By selecting the ratios described, a “creamy powder” composition is advantageously provided. The creamy powder product form provides a particularly pleasant aesthetic while enhancing stability of both color and the UV-filters.

### Dissolved or Finely Dispersed Polymer Component

Compositions of the present invention may include a dissolved or finely dispersed polymer component. As used herein, “dissolved or finely dispersed polymer component” means containing polymers that exist as units in the composition, typically having an average particle size of less than about 0.1 microns, preferably less than about 0.05 microns. The dissolved or finely dispersed polymer component may serve as a film-forming or gelling agent. Suitable examples include those containing film-forming polymers and silicone elastomers that dissolve or finely disperse in the oil component of the composition. Examples of suitable film-forming agents include silicone resin polymers, such as acrylate/dimethicone copolymers, e.g, KP 545L, available as a 40% active in dimethicone, from Shin-Etsu Corporation of Japan; and copolymers of vinyl pyrrolidone and hexadecane, available as GANEX V-216, 96% active material from ISP of Wayne, N.J. Examples of suitable silicone elsatomers include dimethicone/vinyl dimethicone crosspolymer, e.g., available as 9701 cosmetic powder from Dow Corning of Midland, Mich.

The dissolved or finely dispersed polymer component may be present in the composition in a concentration by weight of about 1% to about 6%, such as about 2% to about 6%, preferably about 2% to about 5%.

### Other Functional Ingredients

Compositions of the present invention may include various other functional ingredients that do not compromise the stability of the composition. Preferably the one or more other functional ingredients either dissolve or disperse in the oil component and/or wax component of the composition, or co-blend with the powder component.

For example, the composition may include a safe and effective amount of one or more active ingredients, such as chemically or biologically active ingredients that may be used to treat one or more of various skin conditions including anti-inflammatory ingredients, anti-aging ingredients, anti-acne ingredients, skin-lightening agents, skin-darkening agents, and the like. In one embodiment, the other functional ingredients include an anti-inflammatory like bisabolol. In another embodiment, the other functional ingredients include a preservative such as caprylyl glycol and phenoxyethanol. In another embodiment, the other functional ingredients include encapsulated vitamins such as various forms of Vitamin A, B, C, or E.

The other functional ingredients may be present in the composition in a concentration from about 0.1% to about 12%, such as from about 0.25% to about 10%, preferably from about 0.25% to about 1% by weight of the composition.

In certain embodiments, in order to enhance the safety, asesthetics, and/or stability of the product, the composition is substantially free of volatile solvents (those with a flash point below 30° C., such as ethanol and isopropanol).

Applicant has found that the compositions described herein a high degree of UV-protection, and a particularly pleasant aesthetic while enhancing color stability and chemical stability of the UV-filters.

The compositions of the present invention can be generally prepared by conventional methods known in the cosmetic art. Such methods typically involve mixing of the ingredients in one or more steps to a relatively uniform state, with or without heating, cooling, application of vacuum, and the like. In certain embodiments, methods of making compositions of the present invention include dissolving the organic UV-filter component (including the dibenzoylmethane compound) in one or more oils of the oil component to form a solution of organic UV-filter component in the oil component. This may be done at an elevated temperature such as from about 40° C. to about 70° C. The method may further include melting the wax component and adding the melted wax component to the solution of organic UV-filter component in the oil component to form a preliminary base. The method may further include maintaining the preliminary base at a temperature above the average melting point of all of the waxes in the wax component and adding the color pigment(s) to the preliminary base. The method includes adding the entire powder component at a temperature above the average melting point of all of the waxes. Optional other components may be added, e.g., before or after the powder component. The method may further include pouring the composition into a container or mold, and allowing the composition to cool, such as to form a composition suitable for use as color cosmetic, preferably in the form of a creamy powder.

It is believed that one skilled in the art can, based upon the description herein, utilize the present invention to its fullest extent. The following specific embodiments are to be construed as merely illustrative, and not limitative of the remainder of the disclosure in any way whatsoever. The following non-limiting examples further illustrate the invention.

### EXAMPLES

### Example I

### Preparation of Inventive Examples

The following composition, Inventive Example Ex. 1, shown in Table 1, according to embodiments of the invention described herein was prepared:

TABLE 1
Inventive Example, Ex. 1

| Phase | Trade Name | CTFA Name | % |
|---|---|---|---|
| A | Ganex V-216 | VP/Hexadecene Copolymer | 1.00 |
| A | KP 545L | Dimethicone (and) Acrylate/ | 1.00 |
| | | Dimethicone Copolymer | |
| A | Finsolv TN | C12-15 alkyl benzoate | 5.00 |
| A | Dub Inin B | isononyl isononanoate | 7.40 |
| A | Neo Heliopan HMS | Homosalate | 8.00 |
| A | Neo Heliopan OS | Octisalate (ethylhexyl | 4.00 |
| | | salicylate) | |
| A | Parsol 1789 | Avobenzone | 2.80 |
| A | Neo Heliopan BB | Oxybenzone | 4.00 |
| A | Uvinul N 539 T | Octocrylene | 9.00 |
| A | Corapan TQ | Diethylhexyl | 1.88 |
| | | 2,6-Naphthalate | |
| B | Tinogard TT | Pentaerythrityl | 0.05 |
| | | Tetra-di-t-butyl | |
| | | Hydroxyhydrocinnamate | |
| B | Performer 400 | polyethylene | 2.00 |
| B | white beeswax | beeswax | 6.20 |
| | SP-422P | | |
| C | Sunsphere | Styrene/Acrylates Copolymer | 3.00 |
| C | Ganex V-216 | VP/Hexadecene Copolymer | 0.50 |
| C | Dub Inin B | isononyl isononaoate | 6.15 |
| C | Atlas White | Titanium Dioxide (and) | 7.39 |
| | AS R3435 | Triethoxycaprylylsilane | |
| C | Unipure Yellow LC | Iron Oxide (and) | 1.36 |
| | 182 AS (28201) | Triethoxycaprylylsilane | |
| C | Unipure Red LC 381 | Iron Oxide (and) | 0.36 |
| | AS (28202 | Triethoxycaprylylsilane | |
| C | Unipure | Iron Oxide (and) | 0.21 |
| | Black 989 AS | Triethoxycaprylylsilane | |
| | (R01-94-01) | | |
| D | DC 9701 | Dimethicone/Vinyl | 1.50 |
| | Cosmetic | Dimethicone Crosspolymer | |
| | powder | (and) Silica | |
| D | Dub Inin B | isononyl isononanoate | 4.50 |
| E | Silisphere LS-8H | silica | 4.00 |
| E | Spheron EP-MMS | silica | 8.60 |
| E | Sericite PHN | MICA | 5.05 |
| E | Luxsil | Calcium Aluminum | 4.00 |
| | | Borosilicate | |
| F | Alpha Bisobolol | Bisabolol | 0.30 |
| F | Optiphen | Phenoxyethanol (and) | 0.70 |
| | | Caprylyl Glycol | |
| F | Encapsulated | Polymethyl Methacrylate, | 0.05 |
| | Vitamins | Tocopheryl acetate, | |
| | DCAB5CE | Ascorbic Acid, Pantothenic | |
| | | Acid, Retinyl Palmitate | |
| | | | 100.0 |

Seven additional compositions, Inventive Examples, Ex. 2-8 were also prepared. These compositions were identical to Inventive Example, Ex. 1, except that the concentrations of the three color pigments were adjusted and balanced with

TABLE 2
Inventive Examples Ex. 2-8

| | Atlas | Unipure | Unipure | Unipure | Sercite |
|---|---|---|---|---|---|
| | White | Yellow | Red LC | Black | PHN |
| | AS % | % | % | % | % |
| Ex. 2 | 7.5 | 0.5 | 0.1 | 0.025 | 6.245 |
| Ex. 3 | 7.0 | 0.425 | 0.175 | 0.035 | 6.735 |
| Ex. 4 | 8.7 | 0.825 | 0.225 | 0.05 | 4.570 |
| Ex. 5 | 7.5 | 1.1 | 0.225 | 0.1 | 5.445 |
| Ex. 6 | 7.5 | 1.05 | 0.35 | 0.1 | 5.370 |
| Ex. 7 | 6.5 | 1.3 | 0.3 | 0.125 | 6.145 |
| Ex. 8 | 6.9 | 1.2 | 0.42 | 0.15 | 5.700 |

Inventive Examples, Ex. 1-8 were prepared using the following method. Phase D was prepared by combining each of the ingredients in Phase D and mixing for 10 minutes and allowing the mixture to stand for 24 hours. Phase C was prepared by adding all of the ingredients of Phase C, mixing well, and passing through a triple roller mill 3 times. Phase A was prepared by add all ingredients of Phase A at approximately 50 C into a beaker and mixing until the solids completely dissolve. The ingredients of Phase B were added in order into Phase A at 95 C and mixed until the waxes dissolved. The temperature was held at 95 C, and Phase C was added. The temperature was then reduced to 85 C and Phase D was added to the beaker and mixed well. The temperature of the beaker was decreased to about 80° C., Phase E was then added and mixed well. The ingredients of Phase F were then added in the order shown in Table 1 and allowed to mix for 5 minutes. The compositions were filled into containers 85° C. and allowed to cool.

### Example II

### Inventive Examples and Chemical Stability of UV-Filters in Inventive Example Compositions

Inventive Example, Ex. 9 was prepared in a manner similar to the method described above for Inventive Examples, Ex. 1-8. The following target concentrations for each ingredient are listed in Table 3 below, below:

TABLE 3
Inventive Example, Ex. 9

| Trade Name | CTFA Name | % |
|---|---|---|
| Ganex V-216 | VP/Hexadecene Copolymer | 1.50 |
| Finsolv TN | C12-15 alkyl benzoate | 5.00 |
| Dub Inin B | isononyl isononanoate | 22.41 |
| Hallbrite BHB | Butyloctyl salicylate | 5.00 |
| Neo Heliopan HMS | Homosalate | 8.00 |
| Neo Heliopan OS | Octisalate (ethylhexyl salicylate) | 4.00 |
| Parsol 1789 | Avobenzone | 2.80 |
| Neo Heliopan BB | Oxybenzone | 4.00 |
| Uvinul N 539 T | Octocrylene | 4.00 |
| Corapan TQ | Diethylhexyl 2,6-Naphthalate | 1.88 |
| Performer 400 | polyethylene | 2.00 |
| white beeswax SP-422P | beeswax | 6.20 |
| Sunsphere | Styrene/Acrylates Copolymer | 3.00 |
| Atlas White AS R3435 | Titanium Dioxide (and) | 5.64 |
| | Triethoxycaprylylsilane | |
| Unipure Yellow LC | Iron Oxide (and) | 0.27 |
| 182 AS (28201) | Triethoxycaprylylsilane | |
| Unipure Red LC 381 | Iron Oxide (and) | 0.07 |
| AS (28202 | Triethoxycaprylylsilane | |
| Unipure Black 989 AS | Iron Oxide (and) | 0.03 |
| (R01-94-01) | Triethoxycaprylylsilane | |
| DC 9701 Cosmetic | Dimethicone/Vinyl Dimethicone | 2.00 |
| powder | Crosspolymer (and) Silica | |
| Silisphere LS-8H | silica | 4.00 |
| Spheron EP-MMS | silica | 12.2 |
| Luxsil | Calcium Aluminum Borosilicate | 5.00 |
| Alpha Bisobolol | Bisabolol | 0.30 |
| Optiphen | Phenoxyethanol (and) Caprylyl | 0.70 |
| | Glycol | |
| | | 100.0 |

Three additional compositions, Inventive Examples, Ex. 10-12 were also prepared. These compositions were identical to Inventive Example, Ex. 9, except that the concentrations of the three color pigments were adjusted and balanced with titanium dioxide, as shown in Table 5, below:

TABLE 4
Inventive Examples Ex. 10-12

| | Atlas | | | |
|---|---|---|---|---|
| | White | Unipure | Unipure | Unipure |
| | AS | Yellow | Red LC | Black |
| Ex. 10 | 5.73 | 1.16 | 0.37 | 0.18 |
| Ex. 11 | 6.44 | 1.84 | 0.55 | 0.28 |
| Ex. 12 | 7.16 | 1.45 | 0.46 | 0.23 |

The Inventive Examples were evaluated for chemical stability of organic UV-filters using conventional high performance liquid chromatography (HPLC). A sample of Inventive Example, Ex. 11 (having a target total concentration of iron oxide of 2.67%) was held at 5′C for 4 weeks, while a sample of the same composition was held at 50° C. for 4 weeks. As shown in Table 5, the sample held at 50° C. surprisingly had 96% (=2.70/2.76) of the avobenzone level of the 5′C sample. It should be noted that due to typical, small massing errors in sample preparation, assay (%) at 50° C./assay (%) at 5′C is viewed as a better measure of chemical stability than assay (%) at 50′C/target.

Similar elevated temperature testing was performed for Inventive Example, Ex. 9, 10 and 12. These assays also showed similarly high levels of avobenzone remaining.

TABLE 5
Chemical Stability of Inventive Examples

| | Test Composition | | | | |
|---|---|---|---|---|---|
| | Ex. 11 | Ex. 11 | Ex. 12 | Ex. 9 | Ex. 10 |
| | 4 wk @ | 4 wk @ | 4 wk @ | 4 wk @ | 4 wk @ |
| Assay (%) | 5° C. | 50° C. | 50° C. | 50° C. | 50° C. |
| Octocrylene | 3.89 | 3.86 | 3.92 | 3.88 | 3.92 |
| Octisalate | 4.00 | 3.98 | 4.03 | 3.98 | 4.02 |
| Homosalate | 8.01 | 7.96 | 8.08 | 7.97 | 8.05 |
| Oxybenzone | 4.08 | 3.98 | 4.03 | 4.01 | 4.06 |
| Avobenzone | 2.76 | 2.70 | 2.76 | 2.69 | 2.75 |

### Example III

### UV-Protection of Inventive Example Compositions

Two inventive example compositions, Inventive Examples, Ex. 13 and Ex. 14 having the same formulation as Ex. 1 were tested for sun protection factor (SPF) using a conventional in-vivo static SPF test method. The compositions had an SPF reported as 58.08 and 55.77 respectively.

### Example IV

### Color Stability of Inventive Example Compositions and Comparative Example Compositions

An Inventive Example, Ex. 15 was prepared that was identical to Inventive Example, Ex. 1, except that the concentrations of color pigments, titanium dioxide, and mica are shown below in Table 6.

TABLE 6
Inventive Example, Ex. 15

| | Atlas | | | | |
|---|---|---|---|---|---|
| | White | Unipure | Unipure | Unipure | Sercite |
| | AS | Yellow | Red LC | Black | PHN |
| Ex. 15 | 8.00 | 1.50 | 0.325 | 0.10 | 4.52 |

A Comparative Example, Comp. 1, was prepared identically, except that Phase B, Phase D, and Phase E were omitted. Comparative example, Comp. 1 therefore had no wax component, no powder component, and no silicone elastomer, but maintained the same relative concentrations of the remaining ingredients. Thus Comp. 1 was a highly fluid, thin liquid, unlike Inventive Example, Ex. 15, which had the form of a creamy powder.

These two compositions were evaluated for color stability using the following method. The compositions were each placed in three small plastic pans having a diameter of about 3 cm and a thickness of about 2 mm. A small glass sheet having dimensions of 4.5 cm×4.5 cm×1 mm was placed slowly across the top of the pans in order to remove the air bubbles and to obtain a homogeneous color.

For each test formula L.a.b was measured using a colorimeter for each of the three pans. The mean L, a and b were calculated for each test composition and reported as the “initial” value. Colorimeter testing was also performed on samples that were allowed to age for either 16 days of one month, at either room temperature (RT) or elevated temperature (50° C.). A maximum standard deviation of 0.20 was accepted for each of L, a and b. If the standard deviation for any of the three L, a and b parameters was greater than this amount it was repeated. E is defined as the square root the sum of the squares of L, a, and b. Delta E, a measure of color change due to color instability, is the change in E with respect to the initial value.

The following results were obtained, as shown in Table 7:

TABLE 7
Color Stability Comparison

| Test Composition | | L | a | b | Delta E |
|---|---|---|---|---|---|
| Comp. 1, initial | — | 59.60 | 10.24 | 19.85 | — |
| Comp. 1, 16 days | RT | 59.55 | 9.73 | 19.82 | 0.51 |
| Comp. 1, 16 days | 50° C. | 58.71 | 8.46 | 17.75 | 2.89 |
| Ex. 15, initial | | 60.66 | 9.96 | 17.50 | — |
| Ex. 15, 1 month | 50° C. | 60.81 | 9.83 | 17.37 | 0.24 |

As shown in Table 7, for elevated temperature (50° C.), Inventive Example, Ex. 15 exhibited less than one tenth of the (undesirable) color change in one month than the comparative example showed in about half the time.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. An anhydrous color cosmetic composition comprising:
(1) a cosmetically-acceptable hydrophobic vehicle comprising:
a wax component,
an oil component; and
an organic UV-filter component dissolved in the wax component and the oil component, wherein the organic UV-filter component comprises a dibenzoylmethane compound and is present in a concentration by weight of at least about 15% of the color cosmetic composition; and
(2) a powder component distributed in and substantially insoluble in the hydrophobic vehicle, the powder component comprising
a color pigment present in a concentration by weight of at least about 0.1% of the color cosmetic composition; and
an oil-absorbing powder.

2. The anhydrous color cosmetic composition of claim 1 comprising at least about 0.25% by weight of the color pigment.

3. The anhydrous color cosmetic composition of claim 1, wherein the wax component is present in a concentration by weight of about 2% to about 20% of the color cosmetic composition.

4. The anhydrous color cosmetic composition of claim 1, wherein the oil component is present in a concentration by weight of about 10% to about 40% of the color cosmetic composition.

5. The anhydrous color cosmetic composition of claim 1, wherein the powder component is present in a concentration by weight of about 15% to about 55% of the color cosmetic composition.

6. The anhydrous color cosmetic composition of claim 1, wherein the weight ratio of the cosmetically-acceptable hydrophobic vehicle to the powder component is about 1:1 to about 3:1.

7. The anhydrous color cosmetic composition of claim 1, wherein the weight ratio of the cosmetically-acceptable hydrophobic vehicle to the powder component is about 1.5:1 to about 2.5:1

8. The anhydrous color cosmetic composition of claim 1, wherein the wax component has a weighted average melting point of waxes of about 60-C to about 80-C.

9. The anhydrous color cosmetic composition of claim 1, further comprising a dissolved or finely dispersed polymer component.

10. The anhydrous color cosmetic composition of claim 1, wherein the oil-absorbing powder comprises a highly-oil-absorbing powder.
